(19) United States
(12) Patent Application Publication
(10) Pub. No.: US 2012/0071807 A1
(43) Pub. Date: Mar. 22, 2012

(54) Silver-Silicone Antimicrobial Dressing

(75) Inventor: Robert L. McClure, JR., Elizabethton, TN (US)

(73) Assignee: WEBTEC Converting, LLC., Knoxville, TN (US)

(21) Appl. No.: 12/883,782

(22) Filed: Sep. 16, 2010

(51) Int. Cl. A61L 15/44

(52) U.S. Cl. 602/48

(57) ABSTRACT

An antimicrobial dressing comprising a hydrophobic, porous, meshy, silicone-coated contact layer and a fibrous substrate layer that carries and releases a metal-based antimicrobial agent to prevent infection. In some embodiments, the contact layer comprises a mesh of silicone-coated polyethylene fibers. The silicone in the contact layer helps to reduce the appearance of scars as wounds heal. In some embodiments, the substrate layer comprises a fibrous mesh in which some or all of the fibers are coated in a zeolite carrying metal ions. When the dressing encounters moisture, and moisture penetrates to the substrate layer, the moisture acts to release the antimicrobial metal ions from the zeolites. The ions then migrate through the porous, meshy contact layer into the wound, where they kill microorganisms that cause infection. In some embodiments, the antimicrobial ions carried in the substrate layer are silver ions; or a mixture of silver ions and copper ions; or a mixture of silver ions and zinc ions; or a mixture of silver ions and sodium ions.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

The present invention relates to medicated, antimicrobial, or antibacterial dressings and more particularly to a layered dressing impregnated with and carrying a metal-based antimicrobial agent.

2. Description of the Related Art

Dressings have long been known in the art for protecting and treating wounds or areas of the epidermis that display irritation or visible infection. (Hereinafter, “wounds” is understood to encompass a wide variety of skin injuries and irregularities, including lacerations and puncture wounds as well as, e.g., rashes and eczema.) In recent decades, medical practitioners have come to understand the benefits of including an antimicrobial or anti-infection agent in the dressing to prevent infection of the wound during treatment. A number of devices are known in the art and in related fields, including the following U.S. patents:

| U.S. Pat. No. | Date | | Inventor(s) |
|---|---|---|---|
| 4,561,435 | Dec. | 31, 1985 | McKnight et al. |
| 4,563,184 | Jan. | 07, 1986 | Korol |
| 6,087,549 | Jul. | 11, 2000 | Flick |

| U.S. Pat. No. | Date | | Inventor(s) |
|---|---|---|---|
| 6,093,414 | Jul. | 25, 2000 | Capelli |
| 6,284,941 | Sep. | 04, 2001 | Cox et al. |
| 6,399,091 | Jun. | 04, 2002 | Berthold et al. |
| 6,605,751 | Aug. | 12, 2003 | Gibbins et al. |
| 7,118,761 | Oct. | 10, 2006 | Canada et al. |
| 7,201,925 | Apr. | 10, 2007 | Gillis |
| 7,255,881 | Aug. | 14, 2007 | Gillis et al. |

Devices are known in the art and in related fields also include the following U.S. published patent applications:

| U.S. Pat. Pub. No. | Pub. Date | | Inventor(s) |
|---|---|---|---|
| 2003/0180346 | Sep. | 25, 2003 | Woods |
| 2004/0005364 | Jan. | 08, 2004 | Klein et al. |
| 2004/0030276 | Feb. | 12, 2004 | Flick |
| 2007/0134301 | Jun. | 14, 2007 | Ylitalo et al. |
| 2007/0293799 | Dec. | 20, 2007 | Ma et al. |
| 2009/0104252 | Apr. | 23, 2009 | Alam et al. |

Scarring is a natural part of the healing process. Scar tissue consists mainly of protein collagen formed during the skin's process of wound repair. With the exception of very minor lesions, skin wounds following accidents, disease or surgery all result in some degree of visible scarring. Where the scar tissue is large or in a prominent position on the body, it can be readily apparent to a casual observer and embarrassing or otherwise troubling for the scarred person. It is therefore desirable to have a wound dressing that can help minimize the appearance of scarring during the healing process.

An open wound is at a heightened risk of infection throughout the healing process. In particular, microorganisms such as bacteria and fungi will attempt to establish themselves in the moisture of the exudate extruded from the wound during the healing process. Medical practitioners have discovered that certain metals and metallic compounds, and in particular silver ions, when delivered to a wound, can kill microorganisms within and on the surface of the wound and thereby help fight infection.

It is therefore desirable to have a wound dressing that can supply an antimicrobial agent, such as silver ions, to a wound while also helping the wound to heal in a way to reduce the appearance of scar tissue.

### BRIEF SUMMARY OF THE INVENTION

The present invention includes, in some embodiments, an antimicrobial dressing that incorporates a silicone-based wound contact layer and an antimicrobial or anti-infection agent (hereinafter “antimicrobial agent”) that includes ions of silver or some other metal. In some embodiments, an antimicrobial dressing comprising a hydrophobic, porous, meshy, silicone-coated contact layer and a fibrous substrate layer that carries and releases a metal-based antimicrobial agent to prevent infection. In some embodiments, the contact layer comprises a mesh of silicone-coated polyethylene fibers. The silicone in the contact layer helps to reduce the appearance of scars as wounds heal. In some embodiments, the substrate layer comprises a fibrous mesh in which some or all of the fibers are coated in a zeolite carrying metal ions. When the dressing encounters moisture, and moisture penetrates to the substrate layer, the moisture acts to release the antimicrobial metal ions from the zeolites. The ions then migrate through the porous, meshy contact layer into the wound, where they kill microorganisms that cause infection. In some embodiments, the antimicrobial ions carried in the substrate layer are silver ions. In some embodiments, the substrate layer carries a mixture of silver ions and copper ions. In some embodiments, the substrate layer carries a mixture of silver ions and zinc ions. In some embodiments, the substrate layer carries a mixture of silver ions and sodium ions.

Some embodiments further include a third layer, which adheres to the substrate layer opposite the contact layer, so that when the dressing is applied to a patient's skin, the third or backing layer is the topmost layer. In some embodiments, this backing layer is hydrophobic and acts as a moisture block to prevent the wound from becoming too dry. In some embodiments, the backing layer also includes a second antimicrobial agent, such as silver magnesium phosphate or a zeolite impregnated with silver ions or a mixture of silver ions with zinc or copper ions.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a top-down view of one embodiment of the present invention; and

FIG. 2 is a sectional view of the embodiment shown in FIG. 1.

### DETAILED DESCRIPTION OF THE INVENTION

The present invention, in many embodiments, includes an antimicrobial dressing that incorporates a silicone-based wound contact layer and an antimicrobial or anti-infection agent (hereinafter “antimicrobial agent”) that includes ions of silver or some other metal. In many embodiments, the invention comprises a dressing with at least two layers: a wound contact layer and a substrate layer. The contact layer comprises the portion of the dressing that comes into direct contact with a patient's wound. In some embodiments, the wound contact layer comprises a mesh of fibers, the fibers being coated with a layer of silicone. In some embodiments, the wound contact layer comprises a high-density polyethylene mesh with a silicone coating. When the dressing is applied to a patient's skin, silicone on the wound contact layer reduces the appearance of scar tissue. The silicone-coated wound contact layer is hydrophobic and therefore does not absorb moisture from exudate in the wound.

In some embodiments, the substrate layer, which is moisture-permeable, comprises a fibrous substrate in which some or all of the fibers are coated in a zeolite carrying metal ions. When the dressing encounters moisture, and moisture penetrates to the substrate layer, the moisture acts to release the antimicrobial metal ions from the zeolites. The ions then migrate through the porous, meshy contact layer into the wound, where they kill microorganisms that cause infection. Moisture is necessary for most microbes and infectious organisms to thrive; with the present invention, the presence of that same moisture acts as a trigger to release the antimicrobial agent.

In some embodiments, the antimicrobial ions carried in the substrate layer are silver ions. Silver ions, when introduced into wounds, have been shown to have antimicrobial properties that are useful in preventing and fighting infection. In some embodiments, the substrate layer carries a mixture of silver ions and copper ions. In some embodiments, the substrate layer carries a mixture of silver ions and zinc ions. In some embodiments, the substrate layer carries a mixture of silver ions and sodium ions.

In some embodiments, the substrate layer comprises a fibrous mesh in which the fibers are made from FOSSHIELD® or from the anti-microbial fiber described in U.S. Pat. No. 6,841,244.

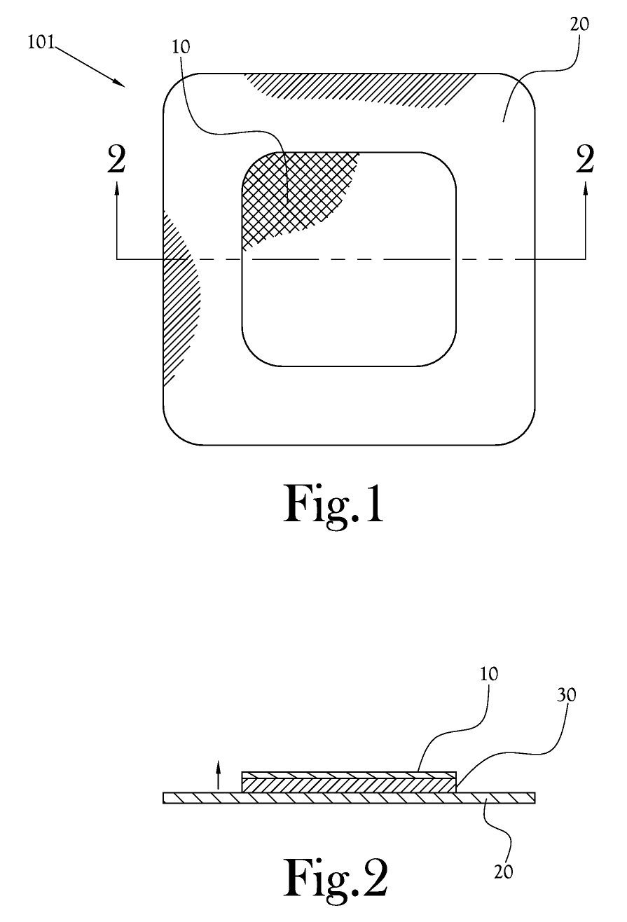

One embodiment of the present invention is illustrated in FIGS. 1 and 2. As shown in FIG. 1, a dressing 101 includes a wound contact layer 10 and a backing layer 20. As shown in the sectional view of FIG. 2, a middle or substrate layer 30 is interposed between the contact layer 10 and the backing layer 20.

In the illustrated embodiment, the contact layer 10 comprises a porous mesh of fibers wherein some or all of the fibers are coated in silicone. When the dressing is used on a person with a wound, the contact layer 10 is placed directly over the wound and comes into physical contact with the wound. The silicone in the contact layer 10 interacts with the wound to reduce the appearance of scar tissue. The contact layer 10 is hydrophobic, and thus moisture passes through the porous mesh of the contact layer 10 without being absorbed.

In the illustrated embodiment, the substrate layer 30 comprises a number of fibers wherein some or all of the fibers are coated with zeolites carrying antimicrobial metal ions. When the dressing is used on a person with a wound, moisture from the wound exudate passes through the porous and meshy contact layer 10 into the substrate layer 30. The moisture acts to release the antimicrobial ions from the zeolites; the ions then migrate within the moisture through the contact layer 10 and into the wound. The direction of ion flow is indicated by the direction arrow in FIG. 2.

The illustrated embodiment includes a backing layer 20. As shown in FIG. 1, the backing layer 20 in some embodiments has a broader surface area than the contact layer 10, and therefore when the dressing is used on a person's skin the backing layer 20 will come into contact with skin surrounding the wound while the contact layer 10 comes into contact with the wound. In some embodiments, the backing layer includes an adhesive that helps the dressing stay in place on the person's skin once the dressing is applied. In some embodiments, the dressing is held in place by other affixing means. In some embodiments, the backing layer is fabricated from a moisture-impermeable or non-porous material and forms a moisture-tight seal between itself and the person's skin. The backing layer thus acts as a moisture shield to keep moisture from the wound from escaping through the substrate layer to be evaporated in the air on the other side of the dressing. Thus the backing layer helps to keep the wound from becoming too dry.

In some embodiments, the backing layer also includes a second antimicrobial agent that supplements the microbe-killing activity of the first antimicrobial agent in the substrate layer. In some embodiment, the second antimicrobial agent is silver magnesium phosphate or a zeolite impregnated with silver ions or a mixture of silver ions with zinc or copper ions. In these embodiments, when moisture penetrates the substrate layer and reaches the backing layer, the moisture releases the second antimicrobial agent from the backing layer; the second antimicrobial agent then migrates within the moisture through the substrate layer and the contact layer into the wound. In this way, the second antimicrobial agent from the backing layer supplements the antimicrobial agent carried in the substrate layer. The direction of flow for the second antimicrobial agent is indicated by the direction arrow in FIG. 2. In some embodiments, the backing layer is a Delnet® mesh that carries one or more antimicrobial agents, such as silver magnesium phosphate or a zeolite impregnated with silver ions or a mixture of silver ions with zinc or copper ions.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

Having thus described the aforementioned invention, I claim:

1. An antimicrobial dressing comprising
a meshy contact layer to contact a wound, said contact layer including silicone;
a moisture-permeable middle layer in contact with said contact layer, said middle layer including a metal-based first antimicrobial agent to permeate the meshy contact layer to contact the wound; and
a moisture-impermeable top layer, said top layer including a second antimicrobial agent to permeate the middle layer and the contact layer to supplement the first antimicrobial agent in contacting the wound.

2. The antimicrobial dressing of claim 1 wherein said first antimicrobial agent is selected from the group consisting of silver, sodium, copper, and zinc.

3. The antimicrobial dressing of claim 1 wherein said first antimicrobial agent is a zeolite carrying metal ions, said metal ions selected from the group consisting of silver, sodium, copper, and zinc.

4. The antimicrobial dressing of claim 1 wherein said first antimicrobial agent is a zeolite carrying a first metal ion and a second metal ion, wherein said first metal ion is silver and said second metal ion is selected from the group consisting of sodium, copper, and zinc.

5. The antimicrobial dressing of claim 1 wherein said moisture-permeable fibrous middle layer comprises fibers coated with zeolites carrying metal ions.

6. The antimicrobial dressing of claim 1 wherein said second antimicrobial agent includes a zeolite carrying silver ions.

7. The antimicrobial dressing of claim 1 wherein said second antimicrobial agent includes a zeolite carrying a mixture of silver ions and zinc ions.

8. The antimicrobial dressing of claim 1 wherein said second antimicrobial agent includes silver magnesium phosphate.

9. A laminar antimicrobial dressing comprising
a fibrous first layer to contact a wound, said first layer including fibers coated with silicone;
a second layer, said second layer including fibers coated with zeolites, said zeolites carrying a metal ion selected from the group consisting of silver, sodium, copper, and zinc; and
a hydrophobic third layer to inhibit the passage of moisture from the second layer to the air.

10. The laminar antimicrobial dressing of claim 9 wherein said zeolites are carrying a mixture of silver ions and copper ions.

11. The laminar antimicrobial dressing of claim 9 wherein said zeolites are carrying a mixture of silver ions and sodium ions.

12. The laminar antimicrobial dressing of claim 9 wherein said zeolites are carrying a mixture of silver ions and zinc ions.

13. The laminar antimicrobial dressing of claim 9 wherein said hydrophobic third layer is carrying a second antimicrobial agent.

14. The laminar antimicrobial dressing of claim 13 wherein said second antimicrobial agent includes a zeolite carrying silver ions.

15. The laminar antimicrobial dressing of claim 13 wherein said second antimicrobial agent includes a zeolite carrying a mixture of silver ions and zinc ions.

16. The laminar antimicrobial dressing of claim 13 wherein said second antimicrobial agent includes silver magnesium phosphate.

17. An antimicrobial dressing comprising
a hydrophobic meshy contact layer to contact a wound, said contact layer including silicone; and
a moisture-permeable fibrous substrate layer, said substrate layer including fibers coated with zeolites, said zeolites being carrying a metal ion selected from the group consisting of silver, sodium, copper, and zinc.

18. The antimicrobial dressing of claim 17 wherein said zeolites are carrying a first metal ion and a second metal ion, wherein said first metal ion is silver and said second metal ion is selected from the group consisting of sodium, copper, and zinc.
